(19) United States
(12) Patent Application Publication
(10) Pub. No.: US 2019/0091112 A1
(43) Pub. Date: Mar. 28, 2019

(54) REMOVER SET

(71) Applicant: MTG CO., LTD., Nagoya-shi (JP)

(72) Inventor: Tsuyoshi MATSUSHITA, Nagoya-shi (JP)

(73) Assignee: MTG CO., LTD., Nagoya-shi (JP)

(21) Appl. No.: 15/845,425

(22) Filed: Dec. 18, 2017

(30) Foreign Application Priority Data
Sep. 28, 2017 (JP) 2017-187292

(51) Int. Cl. A61K 8/02; A45D 33/36; A45D 44/02

(52) U.S. Cl. CPC A61K 8/0204; A45D 33/36; A45D 2200/1063; A61K 8/0279; A61K 2800/47; A45D 44/02

(57) ABSTRACT

Provided is a remover set that allows an attraction head of a remover to easily drop down to the bottom of a holding hollow part of a remover stand, and excels in usability. The remover includes an attraction head having a magnet built therein; a root part connecting to the attraction head; and a grip part extending from the root part. The root part has a recessed portion recessed inward than its vicinity. The remover stand has a holding hollow part including a tip-holding portion that has a concave shape corresponding to a shape including a tip of the attraction head; an opening end part formed upward of the tip-holding portion; and a guide part that is arranged in a portion that is positioned between the tip-holding portion and the opening end part and faces the recessed portion, and is inclined inward as going upward from the tip-holding portion.

### CROSS-REFERENCE

The present application claims priority under 35 U.S.C. § 119 to Japanese Application No. 2017-187292, filed on Sep. 28, 2017, entitled “ REMOVER SET”. The contents of this application are incorporated herein by reference in their entirety.

### BACKGROUND OF THE INVENTION

### Field of the Invention

The present invention relates to a remover set.

### Description of the Related Art

Paste-like cosmetic agents to be in use for cosmetic purposes are classified broadly into two types; one of which has a property of coming into a film-like state after the lapse of time after application on a human skin, and another one has a property of maintaining the paste-like state even after the lapse of time. The cosmetic agents of the type which come into a film-like state after application on the human skin can be easily separated from the human skin by pinching a portion of the cosmetic agent in the film-like state and peeling off the cosmetic agent. On the other hand, as for measures of removing from the human skin the used cosmetic agent of the type maintaining the paste-like state after application on the human skin, it is common to wipe off the cosmetic agent with cotton or the like or to wash off it with lukewarm water or the like.

As a measure of removing the used cosmetic agent from the human skin more easily than these common measures, it is proposed to attract the cosmetic agent applied on the human skin by magnetic attraction force and thereby remove the cosmetic agent from the human skin. For example, Patent Document 1 discloses a remover including a grip part, and an attraction head that is connected to the grip part, and is configured to attract a cosmetic agent that has been applied on a human skin by a magnetic attraction force and thereby remove the cosmetic agent from the human skin.

Patent Document 1 also discloses a remover stand having a head housing portion for housing the attraction head of the remover. This remover stand makes it possible to keep the remover in a holding posture such that the grip part is directed upward and the attraction head is directed downward by inserting the attraction head into the head holding portion in which the remover takes the aforementioned holding posture.

### PRIOR ART LITERATURE

### Patent Document

Patent Document 1

WO2016/084534

When placing the remover on the remover stand, a user inserts the tip of the attraction head of the remover into the head holding portion, and then the user, in some cases, releases its hand(s) from the grip part of the remover until the tip of the attraction head will reach the bottom of the head holding portion. On such an occasion, if the remover takes the aforementioned holding posture, i.e. the posture in which the grip part is directed upward and the attraction head is directed downward, the remover will fall down by its own weight, so that the tip of the attraction head will be naturally inserted into the bottom of the head holding portion.

However, the remover dropping down by its own weight does not always take the aforementioned holding posture. Depending on the posture taken at the time when the user has released its hand(s) from the grip part, the remover may take an inclined posture different from the aforementioned holding posture. If the remover would drop down in such inclined posture(s), the tip of the attraction head tends to be easily caught by the inner wall surface of the head holding portion before reaching the bottom of the head holding portion. As a result, the remover is easily stopped from dropping down, taking the posture inclined from the holding posture and having the tip of the attraction head positioned away from the bottom of the head holding portion.

As described above, in the remover stand disclosed in Patent Document 1, the attraction head may not reach the bottom of the head holding portion, and the remover may be stopped from dropping down with taking an unstable posture inclined from the holding posture. If the remover is stopped from dropping down taking the aforementioned unstable posture, the user is required to correct the posture of the remover into the holding posture so that the attraction head can reach the bottom of the head holding portion to stabilize the posture of the remover. Thus, the remover in Patent Document 1 has room to improve its usability.

The present invention has been made in view of this background, and it is intended to provide a remover set that makes it possible to allow an attraction head of a remover to easily drop down to the bottom of a holding hollow part of a remover stand, and excels in usability.

### SUMMARY OF THE INVENTION

One aspect of the present invention provides a remover set including:

a remover that includes:
an attraction head that has a magnet built therein and is configured to attract a cosmetic agent that has been applied on a human skin by a magnetic attraction force, thereby removing the cosmetic agent from the human skin;
a root part that connects to the attraction head; and
a grip part that extends from the root part; and

a remover stand configured to keep the remover taking a holding posture such that the grip part is directed upward and the attraction head is directed downward; wherein

the root part of the remover has a recessed portion recessed inward than a vicinity of the recessed portion,

the remover stand has a holding hollow part configured to house and hold the attraction head of the remover,

the holding hollow part includes:
a tip-holding portion that is formed inside of the remover stand and has a concave shape corresponding to a shape including a tip of the attraction head of the remover taking the holding posture;
an opening end part that is formed upward of the tip-holding portion, from which the attraction head is inserted; and
a guide part that is arranged in a portion that is positioned between the tip-holding portion and the opening end part and faces the recessed portion, and is inclined inward as going upward from the tip-holding portion.

The remover in the remover set has a recessed portion recessed inward than its vicinity between the grip part and the attraction head. The remover stand has the holding hollow part configured to house and hold the attraction head of the remover. The holding hollow part includes the tip-holding portion that has a concave shape corresponding to a shape including the tip of the attraction head of the remover taking the holding posture, the opening end part that is formed upward of the tip-holding portion, and the guide part that is arranged in a portion that is positioned between the tip-holding portion and the opening end part and faces the recessed portion, and is inclined inward as going upward from the tip-holding portion.

In the remover set, the remover can be placed in the remover stand, for instance, in the following way. Firstly, a user inserts the tip of the attraction head from the opening end part, and then releases its hand(s) from the grip part of the remover to allow the remover to drop down by its own weight. At that time, if the remover takes the holding posture, the tip of the attraction head can reach the bottom of the tip-holding portion.

When the remover takes a posture inclined from an upright state, for instance, in the case where the posture is such that the grip part is inclined to the side of the recessed portion as going upward, it is possible to make the surface of the attraction head on the side of the recessed portion abut on the guide part. Also in the case where the remover takes the aforementioned posture, it is possible to make at least either one of the tip of the attraction head or the surface of the attraction head on the side opposite to the recessed portion abut on the holding hollow part. Thereafter, it is possible to slide the surface of the attraction head along the tip-holding portion to allow the tip of the attraction head to naturally reach the bottom of the tip-holding portion.

On the other hand, when the remover takes a posture inclined reversely to inclination of the aforementioned posture, that is, in the case where the posture is such that the grip part is inclined to the side opposite to the recessed portion as going upward, it is possible to make the surface of the attraction head on the side opposite to the recessed portion abut on the holding hollow part. In the case where the remover takes the aforementioned posture, it is also possible to make at least either one of the tip of the attraction head or the surface of the attraction head on the recessed portion side abut on the guide part. Thereafter, it is possible to slide the tip of the remover under the remover taking the holding posture along the tip-holding portion to allow the tip, the lower end part of the attraction head in the holding posture to naturally reach the bottom of the tip-holding portion.

As described above, the remover set allows the attraction head of the remover to easily reach the bottom of the holding hollow part even in the case where the remover takes the posture inclined from the aforementioned holding posture. Consequently, it is possible to reduce work frequency in correcting the posture of the remover by a user at the time of inserting the remover into the remover stand. As a result, the usability of the remover set for the user can be improved.

### BRIEF DESCRIPTION OF THE DRAWINGS

FIG. 1 is a perspective view of a remover set according to an embodiment.

FIG. 2 is a perspective view of a remover according to the embodiment.

FIG. 3 is an elevation view of the remover according to the embodiment.

FIG. 4 is a rear view of the remover according to the embodiment.

FIG. 5 is a side view of the remover according to the embodiment.

FIG. 6 is a top view of the remover according to the embodiment.

FIG. 7 is a bottom view of the remover according to the embodiment.

FIG. 8 is a sectional view, taken along arrows VIII-VIII in FIG. 5.

FIG. 9 is a sectional view, taken along arrows IX-IX in FIG. 6.

FIG. 10 is an enlarged view of an attraction head in FIG. 9.

FIG. 11 is a bottom view of a case lid portion of the remover according to the embodiment.

FIG. 12 is an illustration of operation for removing a cosmetic agent applied on a human skin from the human skin using the remover according to the embodiment.

FIG. 13 is an illustration of operation for removing a used cosmetic agent adhered to the remover according to the embodiment.

FIG. 14 is a perspective view of a remover stand according to the embodiment.

FIG. 15 is an elevation view of the remover stand according to the embodiment.

FIG. 16 is a rear view of the remover stand according to the embodiment.

FIG. 17 is a side view of the remover stand according to the embodiment.

FIG. 18 is an enlarged view of a projection and its vicinity in FIG. 17.

FIG. 19 is a top view of the remover stand according to the embodiment.

FIG. 20 is a bottom view of the remover stand according to the embodiment.

FIG. 21 is a sectional view, taken along arrows XXI-XXI in FIG. 17.

FIG. 22 is a sectional view, taken along arrows XXII-XXII in FIG. 19.

FIG. 23 is a sectional view, taken along arrows XXIII-XXIII in FIG. 19.

FIG. 24 is a perspective view of a lower part of the remover stand according to the embodiment.

FIG. 25 is a top view of the lower part of the remover stand according to the embodiment.

FIG. 26 is an illustration showing one example of a method for inserting an attraction head of the remover into a holding hollow part of the remover stand (a sectional view corresponding to FIG. 22).

FIG. 27 is an illustration showing one example of a method for inserting the attraction head of the remover into the holding hollow part of the remover stand (a sectional view corresponding to FIG. 22).

### DETAILED DESCRIPTION OF THE INVENTION

In the remover set, the recessed portion may be partly provided at the side periphery surface of the root part of the remover, or may be provided over the whole periphery surface. In the remover stand of the remover set, the tip-holding portion of the holding hollow part may have a tip clearance part dented downward than its vicinity at a position that corresponds to the tip of the attraction head of the remover taking the holding posture such that the grip part is directed upward and the attraction head is directed downward. In this case, it is possible to make the lower end part of the remover, which is inserted into the holding hollow part, project downward from the tip clearance part. Consequently, it is possible to easily position the attraction head inside the holding hollow part, and to settle the posture of the remover more stably.

The remover in the remover set includes the attraction head that is configured to attract a cosmetic agent that has been applied on a human skin by a magnetic attraction force to remove the cosmetic agent from the human skin, the root part that connects to the attraction head, and the grip part that extends from the root part. The attraction head may have a cosmetic agent wiping-off region at the lower end part thereof in the holding posture, i.e. in the posture such that the grip part is directed upward and the attraction head is directed downward. The cosmetic agent wiping-off region generates a magnetic attraction force weaker than the magnetic attraction force generated in its vicinity, or generates no magnetic attraction force.

In the conventional removers as disclosed in Patent Document 1, a magnet is provided on the tip of an attraction head. Accordingly, it is possible to attract the cosmetic agent that has been applied on a human skin with the entire tip of the attraction head by a magnetic attraction force and thereby remove used cosmetic agent from the human skin. On the other hand, because the magnetic attraction force acts also on the cosmetic agent adhered to the surface of the attraction head, the cosmetic agent is easily attracted to the magnet by the magnetic attraction force. As a result, the cosmetic agent tends to remain on the surface of the attraction head even if, for instance, a user tries to collect the used cosmetic agent together on the tip of the attraction head and wipe off it.

In order to avoid such problems and wipe off the cosmetic agent adhered to the surface of the attraction head, for example, it is conceivable to bring the cosmetic agent together into the root part and/or the grip part where no magnetic attraction force acts on. In this case, however, the user is required to grip the attraction head with regripping the remover in order to bring the cosmetic agent to the root part and/or the grip part.

Further, in order not to adhere the used cosmetic agent to the surface of the attraction head, it is conceivable to preliminarily attach a cover such as a cotton sheet or a resin film to the surface of the attraction head and attract the used cosmetic agent through the cover to remove it from the human skin. In this case, however, it is necessary to prepare such a cover preliminarily and attach it to the attraction head.

As seen from above, in the remover disclosed in Patent Document 1, there is a possibility that removal of the used cosmetic agent adhered to the surface of the attraction head may be made troublesome.

To cope with such a problem, it is possible to provide the cosmetic agent wiping-off region at the lower end part of the attraction head under the remover taking the holding posture, i.e. at the part on the opposite side to the grip part to thereby remove the cosmetic agent from the surface of the remover more simply and more easily. Namely, because the cosmetic agent wiping-off region is provided at the lower end part of the attraction head, the user can collect the used cosmetic agent together on the cosmetic agent wiping-off region without regripping the grip part.

Further, the cosmetic agent wiping-off region generates a magnetic attraction force weaker than the magnetic attraction force generated in the vicinity of the cosmetic agent wiping-off region, or generates no magnetic attraction force. It makes it possible to easily wipe off the cosmetic agent that has been collected on the cosmetic agent wiping-off region so as to remove the cosmetic agent from the surface of the remover.

In this way, because the cosmetic agent wiping-off region is provided at a specific position, i.e. at the lower end part of the attraction head under the remover taking the holding posture, it is possible to remove the used cosmetic agent from the surface of the remover more simply and more easily.

As a method of forming the cosmetic agent wiping-off region at the lower end part of the attraction head, various measures can be utilized. For example, a soft-magnetic material can be provided between the magnet and the inside surface of the cosmetic agent wiping-off region in the attraction head. In this case, the soft-magnetic material can decrease or shield the magnetic attraction force generated by the magnet. Consequently it is made possible to form the cosmetic agent wiping-off region at the lower end part of the attraction head.

The magnet may be provided at the position apart from the inside surface of the cosmetic agent wiping-off region in the attraction head. It is made possible to decrease the magnetic attraction force generated by the magnet also by increasing the distance from the magnet to the cosmetic agent wiping-off region. Thus, it is possible to form the cosmetic agent wiping-off region at the lower end part of the attraction head. In order to avoid increase in number of parts and simplify assembly work for the remover, the magnet is preferably arranged at the position apart from the inside surface of the cosmetic agent wiping-off region in the attraction head.

The attraction head of the remover may include:

a pair of main wall portions facing each other;

a peripheral wall portion that connects peripheral edges of the pair of main wall portions with each other; and

the magnet that is arranged between the pair of main wall portions such that an inside surface of each main wall portion faces a magnet pole.

A lower half part of the remover taking the holding posture may include:

a mountain-shaped curved portion having a convexly curved contour on a left side in a plan view on one of the pair of main wall portions;

an S-shaped curved portion having an S-shaped contour that is recessed inward, expands outward, and is recessed inward again as going downward from upward on a right side in the plan view; and

a tip portion that is provided between the mountain-shaped curved portion and the S-shaped curved portion and has a convexly curved contour with a curvature radius smaller than that of any portion of the mountain-shaped curved portion and the S-shaped curved portion.

The tip-holding portion of the holding hollow part of the remover stand may have a hollowed shape corresponding to a shape including a lower half part of the mountain-shaped curved portion, a lower half part of the S-shaped curved portion, and the tip portion as a lower end of the remover taking the holding posture.

The guide part may be arranged between a part of the tip-holding portion, which faces the S-shaped curved portion, and the opening end part.

In the remover set, a remover and a remover stand each having a specific shape as described above are used in combination, so that the attraction head of the remover can be inserted more easily so as to reach the bottom of the holding hollow part of the remover stand even in the case where the posture of the remover is inclined from the holding posture.

The holding hollow part of the remover stand may have an enlarged part between a part of the tip-holding portion, which faces the mountain-shaped curved portion, and the opening end part, the enlarged part having a shape of being enlarged as going upward. In this case, it is possible to make the mountain-shaped curved portion and the enlarged part abut on each other after inserting the attraction head into the holding hollow part, and smoothly slide the mountain-shaped curved portion along the enlarged part. In this way, it is possible to insert the tip portion of the remover so as to easily reach the bottom of the tip-holding portion.

The opening end part may have a sloping shape in which a height of the opening end part on a side corresponding to the S-shaped curved portion is higher than that on a side corresponding to the mountain-shaped curved portion. In this case, the guide part can be formed over a larger area. Thus, it is possible to make a part constituting the S-shaped curved portion and a part constituting the tip portion in the peripheral wall portion more easily abut on the guide part after the attraction head has been inserted into the holding hollow part. As a result, the tip portion of the remover can easily reach the bottom of the tip-holding portion.

### Embodiment

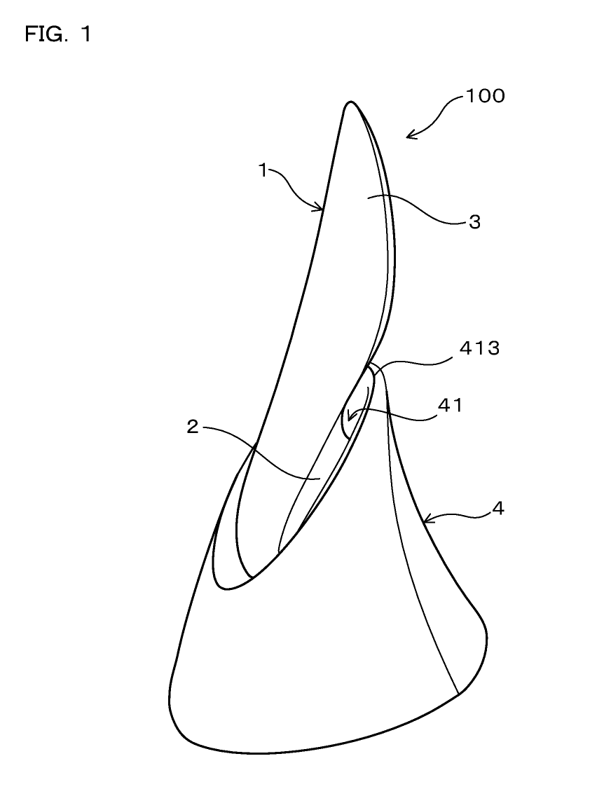

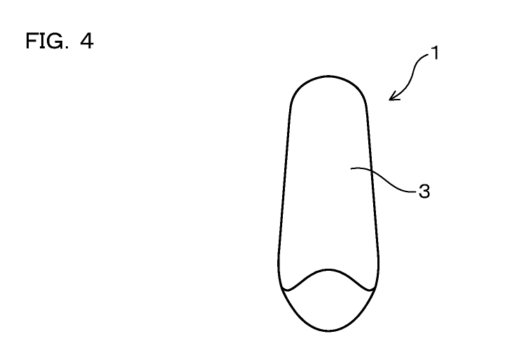

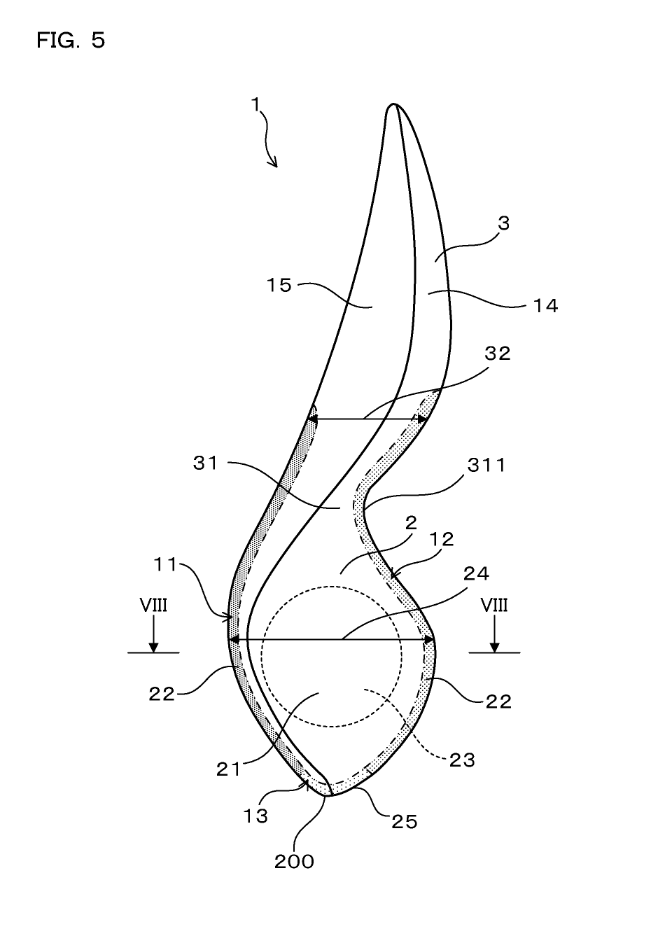

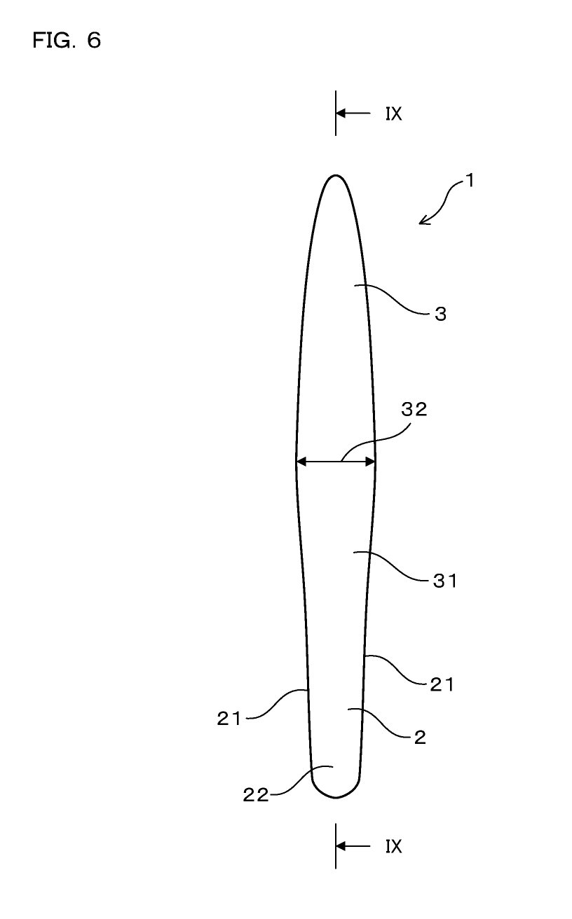

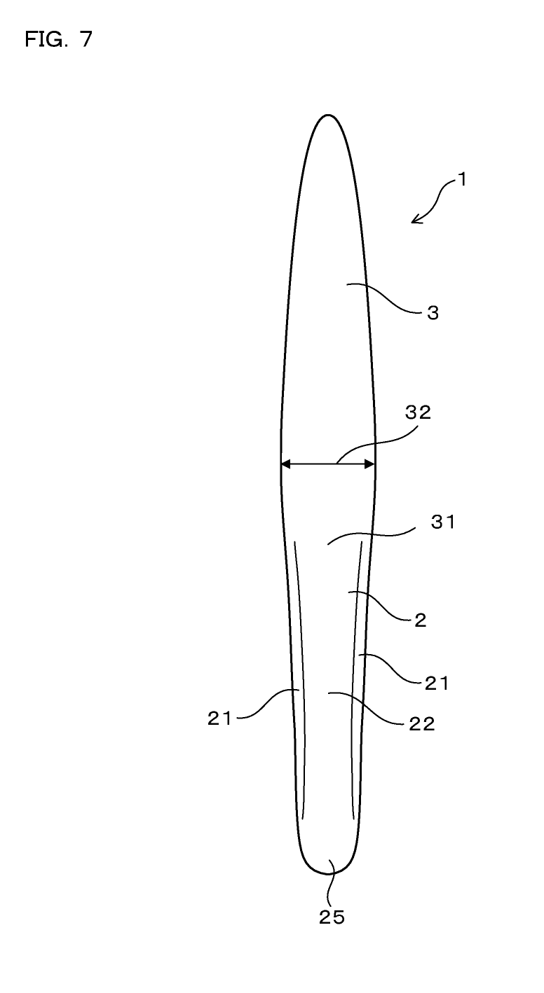

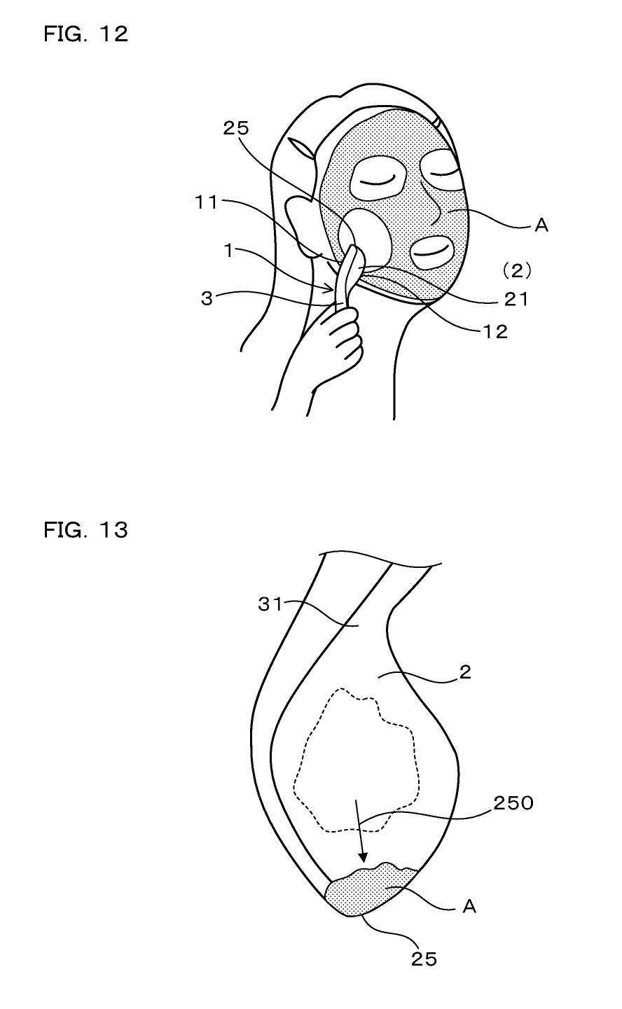

An embodiment of the remover set will be described with reference to FIGS. 1 to 27. As shown in FIG. 1, a remover set 100 includes a remover 1 and a remover stand 4. The remover 1, as shown in FIGS. 5 and 12, includes an attraction head 2 that has a magnet 23 built therein and is configured to attract a cosmetic agent A that has been applied on a human skin by a magnetic attraction force to thereby remove the cosmetic agent from the human skin, a root part 31 that connects to the attraction head 2, and a grip part 3 that extends from the root part 31. The remover stand 4, as shown in FIGS. 1, 17, 22 and 23, is configured to be able to keep the remover 1 taking a holding posture, i.e. a posture such that the grip part 3 is directed upward and the attraction head 2 is directed downward.

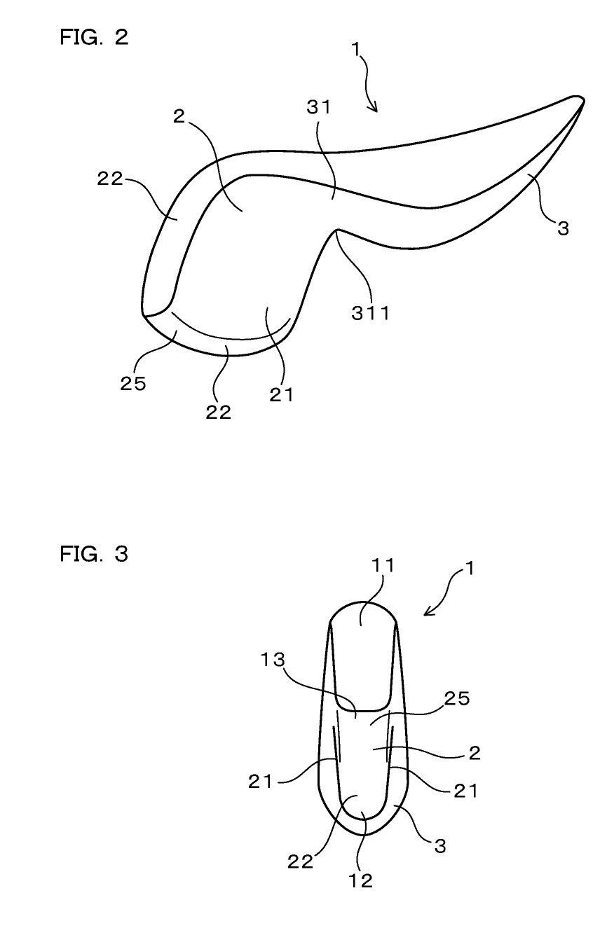

The root part 31 of the remover 1, as shown in FIGS. 2, 5 and 9, has a recessed portion 311 recessed inward than the vicinity of the recessed portion 311.

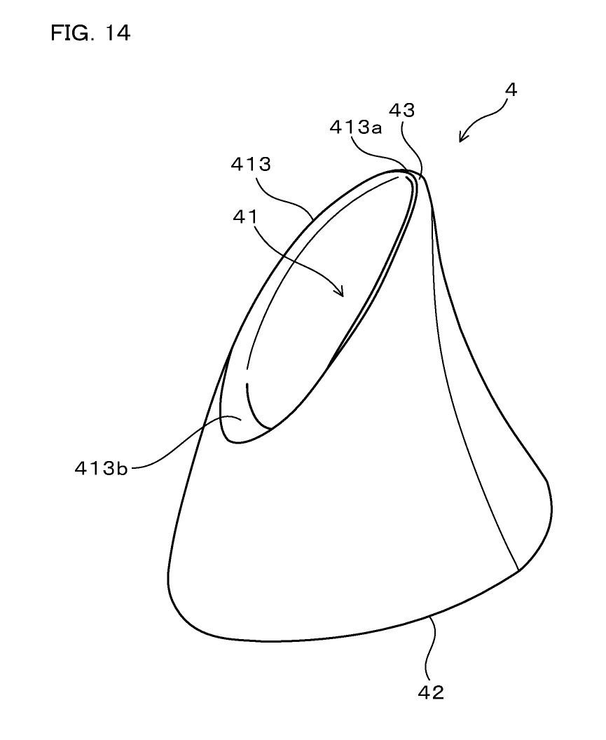

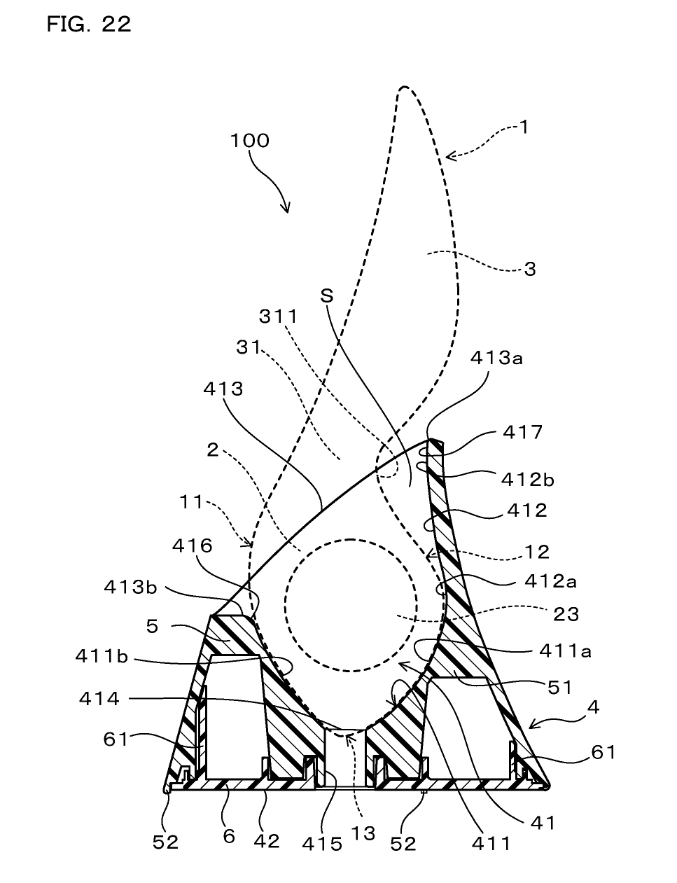

The remover stand 4, as shown in FIGS. 14, 15, 19, and 21 to 23, has a holding hollow part 41 configured to house and hold the attraction head 2 of the remover 1. The holding hollow part 41, as shown in FIG. 22, includes a tip-holding portion 411, a guide part 412 and an opening end part 413.

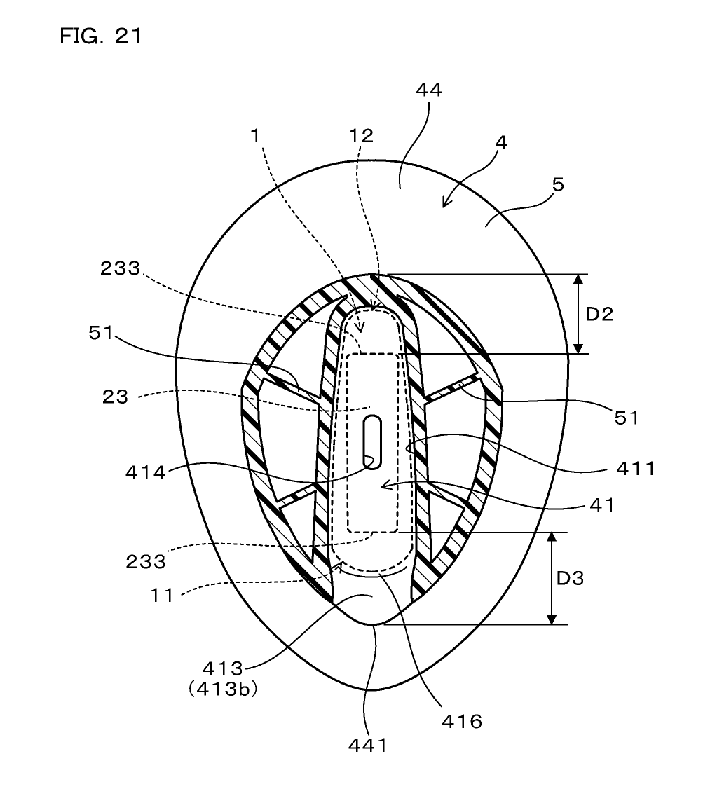

As shown in FIGS. 21 to 23, the tip-holding portion 411 is formed at the bottom of the holding hollow part 41 inside of the remover stand 4, and has a hollowed shape corresponding to the shape of a lower half part of the remover 1 taking the holding posture. The tip-holding portion 411 can hold a lower half part of the attraction head 2.

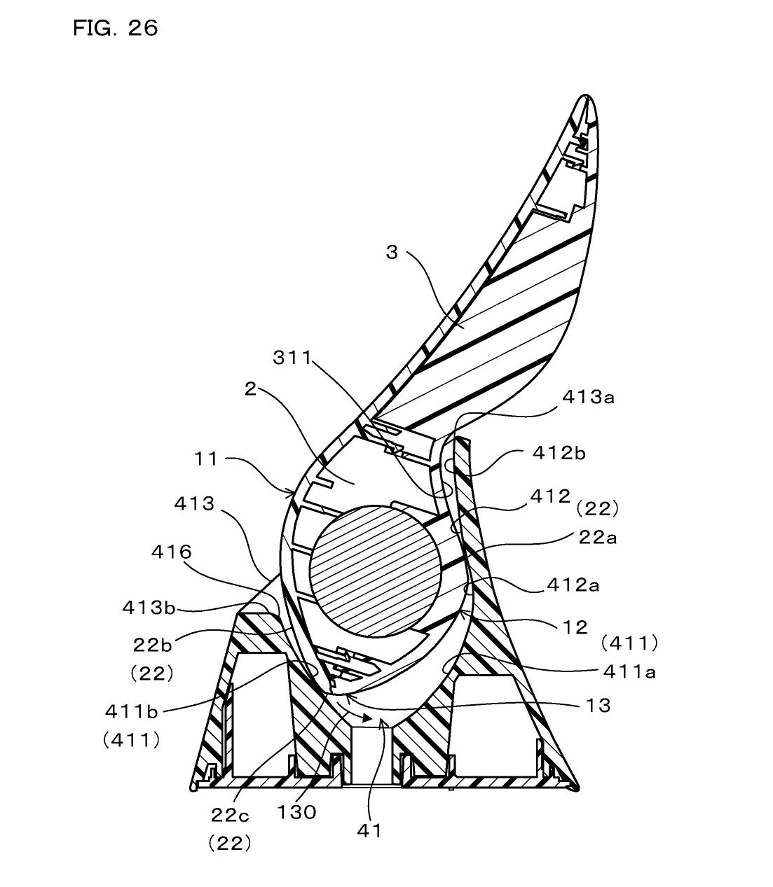

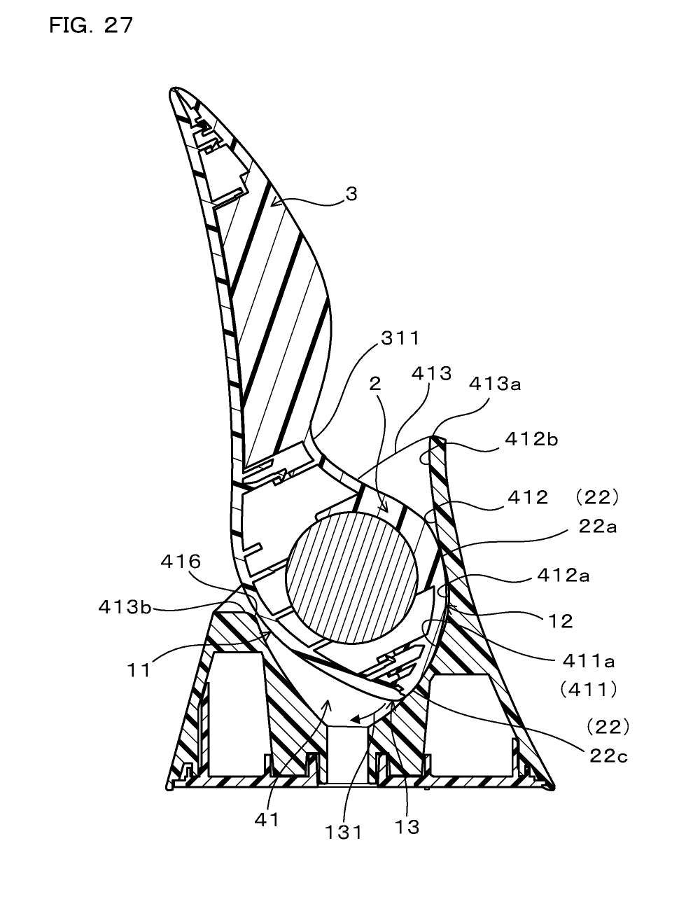

As shown in FIGS. 14, 15, 17, 19, and 22 to 23, the opening end part 413 is formed upward of the tip-holding portion 411. The attraction head 2 is inserted into the holding hollow part 41 from the opening end part 413 as shown, for example, in FIGS. 26 and 27. As shown in FIG. 22, the guide part 412 is arranged in a portion that is positioned between the tip-holding portion 411 and the opening end part 413 and faces the recessed portion 311, and is inclined inward as going upward from the tip-holding portion 411.

Hereinafter, configurations of the remover 1 and the remover stand 4 will be described in detail.

As shown in FIGS. 2, and 5 to 7, the remover 1 is formed in a substantially rod-like shape, and includes the attraction head 2 and the grip part 3 positioned side by side in its longitudinal direction. Hereinafter, in some cases, a side of the attraction head 2 in the longitudinal direction of the remover 1 is referred to as “forward”, and a side of the grip part 3 is referred to as “backward”. Such descriptions regarding the forward and backward directions of the remover 1 will be used in for convenience sake, and have no relation to the direction of the remover 1 under operation of removing the cosmetic agent A from the user's skin, and/or the direction of the remover 1 taking the holding posture.

As shown in FIG. 5, the forward half part of the remover 1, i.e. the part that is continuous from a front end 200 of the attraction head 2 to the root part 31 has a shape recessed inward at the root part 31 and expanded outward at the attraction head 2.

More specifically, as shown in FIG. 5, the lower half part of the remover 1 under the remover 1 taking the holding posture includes a mountain-shaped curved portion 11 having a convexly curved contour on a left side in a plan view on one of a pair of main wall portions 21. In addition, the lower half part of the remover 1 includes an S-shaped curved portion 12 having an S-shaped contour that is recessed inward, expands outward, and is recessed inward again as going downward from upward on a right side in the plan view. Further, the lower half part of the remover 1 includes a tip portion 13 that is provided between the mountain-shaped curved portion 11 and the S-shaped curved portion 12 and has a convexly curved contour with a curvature radius smaller than that of any portion of the mountain-shaped curved portion 11 and the S-shaped curved portion 12. An upper half part of the S-shaped curved portion 12 in the holding posture, i.e. the part that is recessed inward and then expands outward as going downward from upward, forms the recessed portion 311 of the root part 31.

As shown in FIGS. 3 to 7, the backward half part of the remover 1, i.e. the part positioned backward from the root part 31 as the root of the attraction head 2 in the grip part 3 has an outer diameter that becomes gradually larger as going backward from the root part 31, and is the largest at an intermediate part 32 positioned between the root part 31 and the rear end of the grip part 3. The outer diameter of the grip part 3 becomes gradually smaller as going backward from the intermediate part 32.

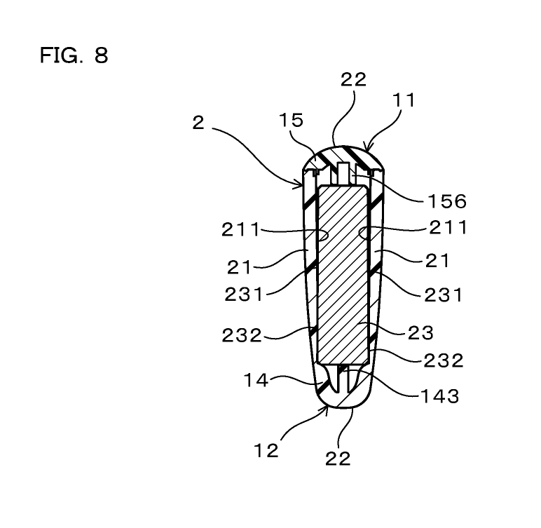

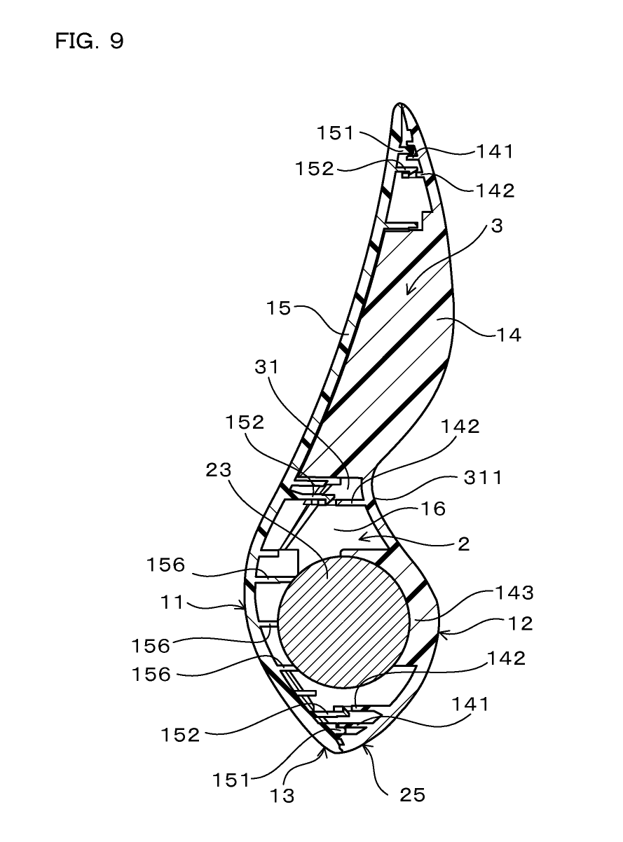

As shown in FIGS. 2, 5, and 9, the remover 1 has the attraction head 2 formed forward thereof. The attraction head 2 includes the pair of main wall portions 21, a peripheral wall portion 22 that connects peripheral edges of the pair of main wall portions 21 with each other, and the magnet 23 that is arranged between the pair of main wall portions 21. The magnet 23, as shown in FIG. 9, is arranged substantially at the center of the attraction head 2 in a plan view from a thickness direction thereof, i.e. a plan view on the main wall portion 21. The magnet 23 of the present embodiment has a disk-like shape, and is magnetized in its thickness direction. As shown in FIG. 8, a pair of end faces 232 serving as a magnet pole 231 of the magnet 23 faces respectively an inside surface 211 of the main wall portions 21.

As shown in FIG. 5, the attraction head 2 of the present embodiment has a width that gradually broadens as going forward from the root part 31, i.e. as going toward the tip portion 13 side in the remover 1 in a plan view from its thickness direction. Further, the attraction head 2 has a largest width part 24 between the root part 31 and the front end 200 of the attraction head 2, the width of which is the largest in a plan view from its thickness direction.

The forward half part of the attraction head 2, i.e. the part positioned forward from the largest width part 24 has a tapered shape the width of which is gradually tapered as going forward. The attraction head 2, as shown in FIGS. 3 and 8, has the largest thickness on a mountain-shaped curved portion 11 side, and the thickness becomes gradually thinner as approaching an S-shaped curved portion 12 side.

The attraction head 2 of the present embodiment generates the strongest magnetic attraction force substantially at the center of the main wall portion 21 in a plan view from its thickness direction, i.e. at the part that faces the end face 232 of the magnet 23. Therefore, the cosmetic agent A that has been applied on the human skin can be attracted by the magnetic attraction force and removed from the human skin by bringing the main wall portion 21 close to the cosmetic agent A as shown in FIG. 12. It is noted that the diameter of the magnet 23 of the present embodiment is 30 mm, and the thickness is 8 mm. The magnitude of the surface magnetic flux density is 220 mT or more at the center of the pair of end faces 232.

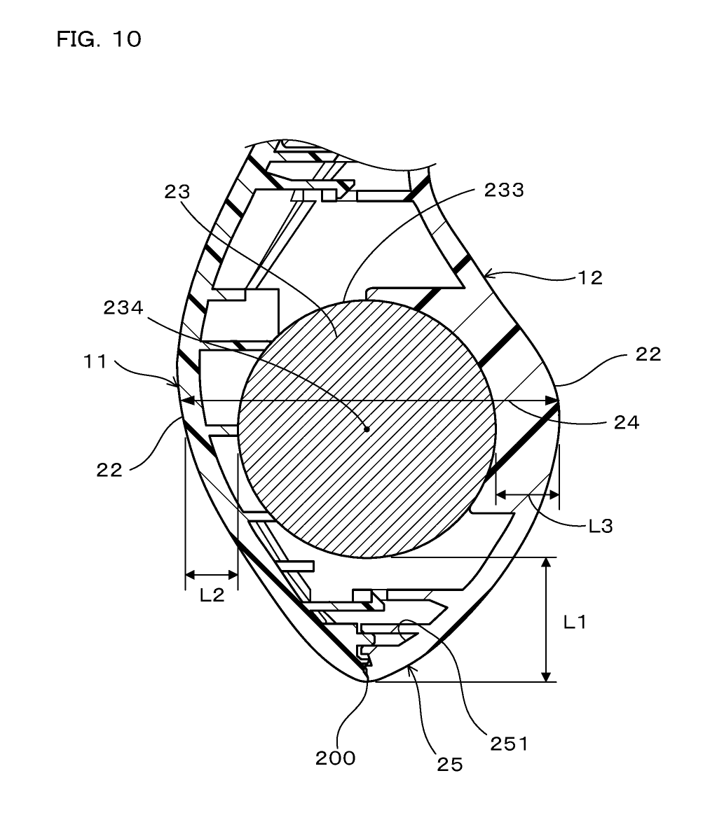

As shown in FIGS. 5 and 9, the attraction head 2 has a cosmetic agent wiping-off region 25 at the lower end part thereof under the remover taking the holding posture (i.e. at the front end 200 and its vicinity). The cosmetic agent wiping-off region 25 generates a magnetic attraction force weaker than that generated in the vicinity of the cosmetic agent wiping-off region 25. In the remover 1 of the present embodiment, as shown in FIG. 10, the magnet 23 is arranged at the position apart from the inside surface 251 of the cosmetic agent wiping-off region 25 in the peripheral wall portion 22 so as to decrease the magnetic attraction force to be generated at the front end of the attraction head 2 as compared to the magnetic attraction force to be generated in its vicinity, and thus the cosmetic agent wiping-off region 25 is formed at the front end of the attraction head 2.

As shown in FIG. 10, the distance from a side peripheral surface 233 of the magnet 23 to an outer surface of the peripheral wall portion 22 of the attraction head 2 becomes gradually shorter as going forward from the root part 31. The distance becomes the shortest in the vicinity of the largest-width part 24, and then becomes gradually longer as approaching the front end 200 of the attraction head 2.

A distance L1 from the side peripheral surface 233 of the magnet 23 to the front end 200 of the attraction head 2 in the longitudinal direction is 14.3 mm. A distance L2 from the side peripheral surface 233 of the magnet 23 to the mountain-shaped curved portion 11 measured on the straight line passing through a center 234 of the magnet 23 and extending in the direction perpendicular to the longitudinal direction is 6.1 mm. A distance L3 from the side peripheral surface 233 of the magnet 23 to the S-shaped curved portion 12 measured on the straight line passing through the center 234 of the magnet 23 and extending in the direction perpendicular to the longitudinal direction is 7.3 mm.

In the remover 1 of the present embodiment, the distance from the magnet 23 to the outer surface of the peripheral wall portion 22 is set as described above, so that the magnetic attraction force is decreased at the cosmetic agent wiping-off region 25 as compared to the magnetic attraction force generated in its vicinity. Consequently, the cosmetic agent adhered to the cosmetic agent wiping-off region 25 can be easily wiped off and removed from the attraction head 2. In the vicinity of the largest-width part 24 in the peripheral wall portion 22, a magnetic attraction force that is stronger than that generated in the cosmetic agent wiping-off region 25 is generated. Therefore, the cosmetic agent A that has been applied on the human skin can be attracted by the magnetic attraction force and removed from the human skin by bringing the vicinity of the largest-width part 24 close to the cosmetic agent A.

As shown in FIGS. 5 and 9, an outer wall of the attraction head 2 and the grip part 3 in the remover 1 is composed of a case base portion 14 that includes the S-shaped curved portion 12 and is extended over the whole in the longitudinal direction of the remover 1, and a case lid portion 15 that includes the mountain-shaped curved portion 11 and is extended over the whole in the longitudinal direction of the remover 1. As shown in FIG. 9, an internal space 16 for housing the magnet 23 is formed between the case base portion 14 and the case lid portion 15.

As shown in FIG. 9, the case base portion 14 has two bosses 141 provided on the end parts in the longitudinal direction, and three claw receiving parts 142 provided on the end parts in the longitudinal direction and on the root part 31. In addition, as shown in FIGS. 8 and 9, a magnet receiving part 143 for holding the magnet 23 is provided at the front end of the case base portion 14.

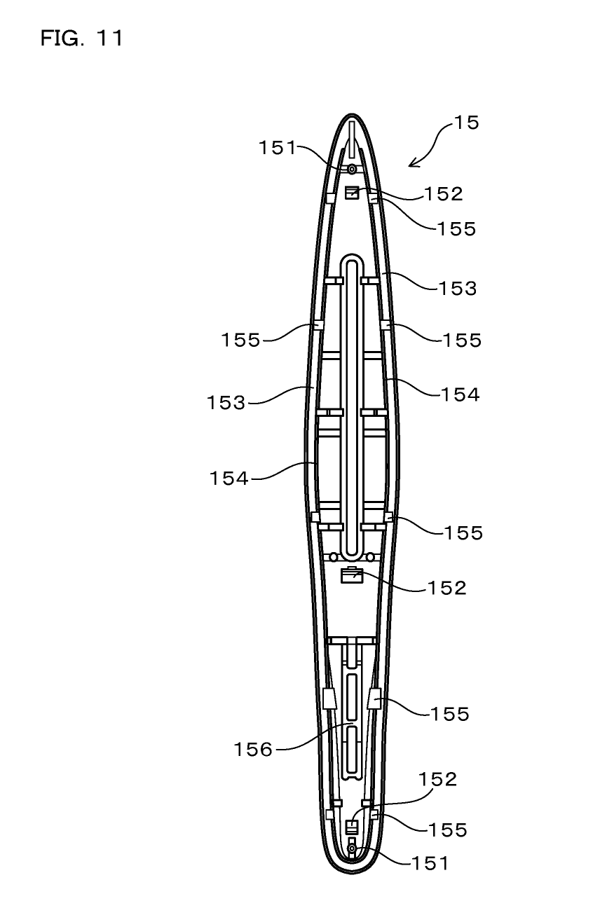

As shown in FIGS. 9 and 11, the case lid portion 15 has two positioning projections 151 provided on the end parts in the longitudinal direction, and three engaging claws 152 provided on the end parts in the longitudinal direction and on the root part 31. As shown in FIG. 9, the positioning projections 151 are inserted into the bosses 141 provided on the case base portion 14. The engaging claws 152 are engaged with the claw receiving parts 142 provided on the case base portion 14.

As shown in FIG. 11, an outer peripheral groove 153 is formed on the outer peripheral edge of the case lid portion 15. Although not shown in a figure, an adhesive material for bonding the case lid portion 15 and the case base portion 14 is held in the outer peripheral groove 153. The outer peripheral edge of the case lid portion 15 and the outer peripheral edge of the case base portion 14 are bonded to each other through the adhesive material.

Ten notches 155 are provided on an inside wall 154 of the outer peripheral groove 153. According to such a configuration, the adhesive material can be released from the notches 155 into the internal space 16 when the case lid portion 15 and the case base portion 14 are combined with each other, to thereby prevent the adhesive material from leaking outside from the case.

As shown in FIGS. 8, 9 and 11, a magnet pressing part 156 for pressing the magnet 23 to a magnet receiving part 143 side in the case base portion 14 is provided on the front end part of the case lid portion 15. As shown in FIGS. 8 and 9, the magnet 23 built in the attraction head 2 is held between the magnet receiving part 143 of the case base portion 14 and the magnet pressing part 156 of the case lid portion 15.

Next, the configuration of the remover stand 4 will be described.

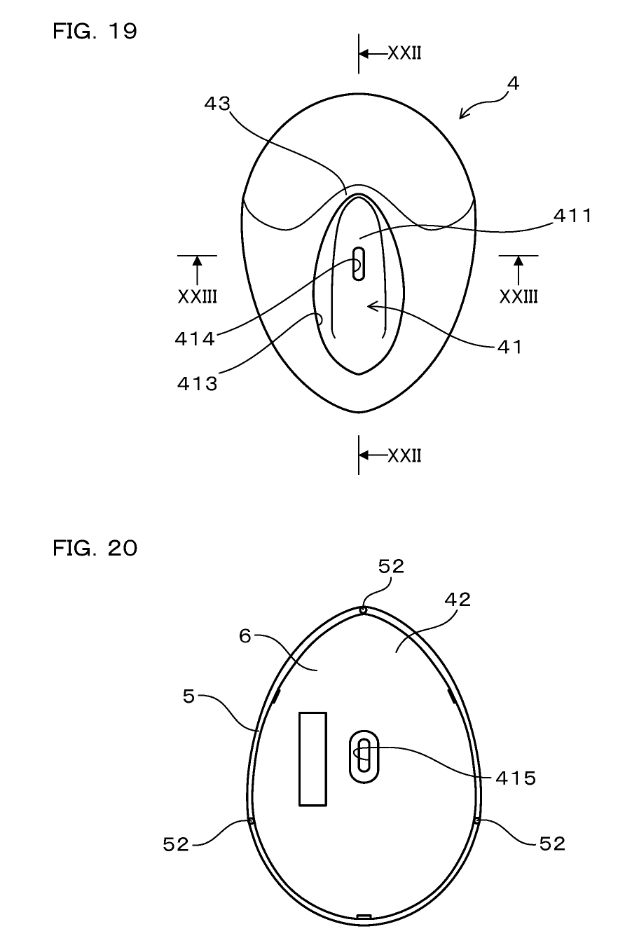

The remover stand 4, as shown in FIGS. 14 to 17, has a approximately truncated cone shape the outside dimension of which becomes gradually smaller as going upward from a bottom surface 42. The bottom surface 42 of the remover stand 4 has an approximately ellipse shape as shown in FIG. 20. Further, as shown in FIG. 19, the remover stand 4 has the holding hollow part 41 for keeping the remover 1 taking the holding posture such that the grip part 3 is directed upward and the attraction head 2 is directed downward (refer to FIGS. 1, 22 and 23).

As shown in FIG. 22, the holding hollow part 41 includes the tip-holding portion 411 formed at the bottom thereof, and the opening end part 413 at the upper end thereof. The tip-holding portion 411 of the present embodiment has a hollowed shape corresponding to a shape including a lower half part of the mountain-shaped curved portion 11, a lower half part of the S-shaped curved portion 12, and the tip portion 13 as a lower end of the remover taking the holding posture, i.e. a posture such that the grip part 3 is directed upward and the attraction head 2 is directed downward. The guide part 412 is arranged between a part 411a of the tip-holding portion 411 which faces the S-shaped curved portion 12, and the opening end part 413. An enlarged part 416 having a shape of being enlarged as going upward, are arranged between a part 411b of the tip holding portion 411 which faces the mountain-shaped portion 11, and the opening end part 413.

As shown in FIGS. 21 to 23, the tip-holding portion 411 has a hollowed shape that corresponds to the lower half part of the attraction head 2 in the holding posture. As shown in FIG. 22, a tip clearance part 414 dented downward than its vicinity is formed in the tip-holding portion 411 at the position corresponding to the tip portion 13 of the remover 1. The tip-holding portion 411 of the present embodiment has a cylindrical part 415 extended downward from the tip clearance part 414 and opened to the bottom surface 42 of the remover stand 4.

The tip-holding portion 411, as shown in FIG. 22, makes it possible to protrude the tip portion 13 of the remover 1 downward from the tip clearance part 414, and as shown in FIGS. 21 to 23, makes it possible to hold the attraction head 2 substantially without any clearance with the lower half part of the attraction head 2.

As shown in FIG. 22, the guide part 412 is connected upward of the part 411a that corresponds to the S-shaped curved portion 12 in the tip-holding portion 411. The guide part 412 of the present embodiment includes a lower end 412a as boundary with the part 411a that corresponds to the S-shaped curved portion 12 in the tip-holding portion 411, and an upper end 412b as a position to be faced to the root part 31 of the remover 1, and is arranged over the lower end 412a to the upper end 412b.

The guide part 412 is gradually inclined inward, i.e. toward the side on which the remover 1 is placed as going upward from the lower end 412b. In the cross section shown in FIG. 22, i.e. in the cross section of the remover stand 4 cut along a longitudinal direction of the bottom surface 42 through the center thereof, the contour of the guide part 412 has an arc shape with a curvature convexed inward. An inclination of the guide part 412 is largest at the upper end 412b, putting the basis on the lower end 412a.

An upward part 417 positioned upward of the guide part 412 in the holding hollow part 41 is inclined gradually outward as going upward from the upper end 412b of the guide part 412, and is connected to the opening end part 413. In the cross section shown in FIG. 22, the contour of the upward part 417 positioned upward of the guide part 412 has an arc shape with a curvature convexed inward.

In the remover set 100 of the present embodiment, a space S is produced between the S-shaped curved portion 12 and the guide part 412 in the state that the remover 1 is kept in the remover stand 4.

As shown in FIG. 22, the enlarged part 416 is connected upward of the part 411b that corresponds to the mountain-shaped curved portion 11 in the tip-holding portion 411. The enlarged part 416 of the present embodiment has smoothly curved surface and has a shape of being enlarged as going upward. In the cross section shown in FIG. 22, i.e. in the cross section of the remover stand 4 cut along the longitudinal direction of the bottom surface 42 through the center thereof, the contour of the enlarged part 416 has an arc shape with a curvature convexed upward.

As shown in FIGS. 14, 15, 17, and 22, the opening end part 413 for inserting the attraction head 2 is formed at the upper end of the holding hollow part 41. As shown in FIG. 22, the opening end part 413 is connected to the enlarged part 416, the parts of the tip-holding portion 411 which correspond to the pair of main wall portions 21 (not shown in a figure), and the guide part 412, respectively. The opening end part 413 of the present embodiment has a sloping shape in which a height of the opening end part on a side 413a corresponding to the S-shaped curved portion 12 is higher than that on a side 413b corresponding to the mountain-shaped curved portion 11.

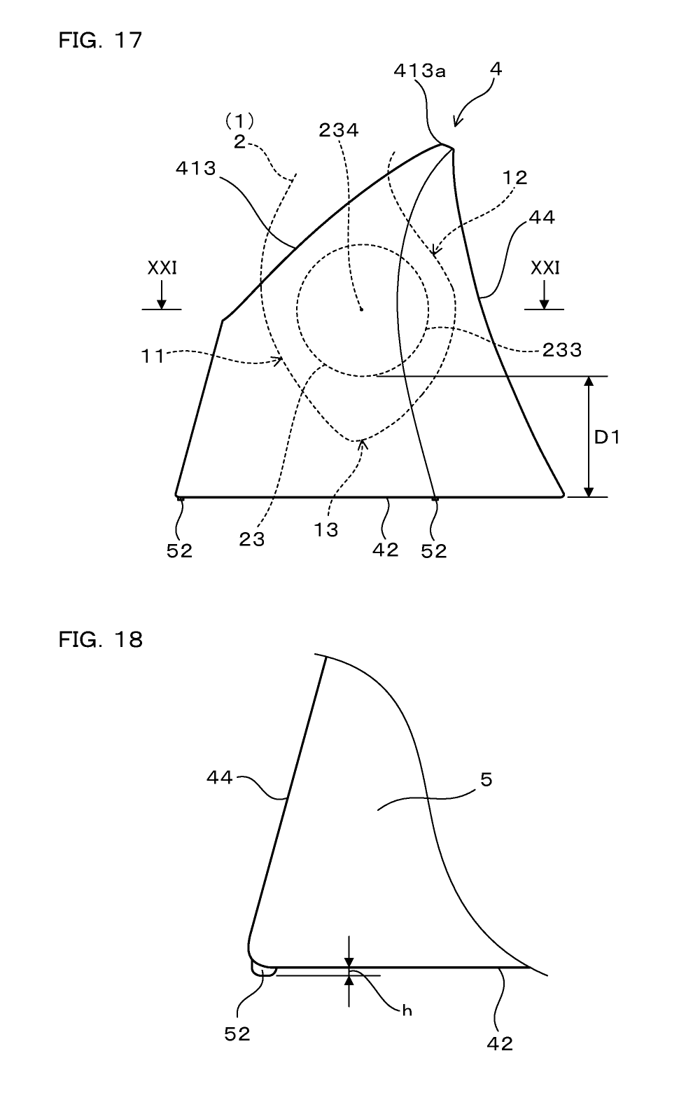

The remover stand 4 under keeping the remover 1 is configured such that the distance from the magnet 23 built in the attraction head 2 to the outer surface of the remover stand 4 is made sufficiently large so as to decrease the magnet flux generated from the magnet 23 in the remover 1. Specifically, in the state that the remover 1 is held in the remover stand 4 of the present embodiment, a distance D1 from the side peripheral surface 233 of the magnet 23 to the bottom surface 42 of the remover stand 4 measured on the straight line passing through the center 234 of the magnet 23 and extending vertically downward is 26.7 mm (refer to FIG. 17). When the remover 1 is held in the remover stand 4 of the present embodiment, the magnetic flux density generated by the magnet 23 is 50 mT or less at any position on the bottom surface 42 of the remover stand 4.

A distance D2 from the side peripheral surface 233 of the magnet 23 to a side surface 44 of the remover stand 4 measured on the horizontal line passing through the center 234 of the magnet 23 and extending to the S-shaped curved portion 12 side is 3.4 mm (refer to FIG. 21). It is noted that the horizontal line passes through the position shifted upward from the side surface 44 of the remover stand 4 under the horizontal line passing through the center 234 of the magnet 23 and extending to the mountain-shaped curved portion 11 side. Therefore, although the distance from the side peripheral surface 233 of the magnet 23 to the remover stand 4 cannot be accurately defined, a distance D3 from the side peripheral surface 233 of the magnet 23 to a boundary 441 between the opening end part 413 and the side surface 44 in the horizontal direction is 13.4 mm.

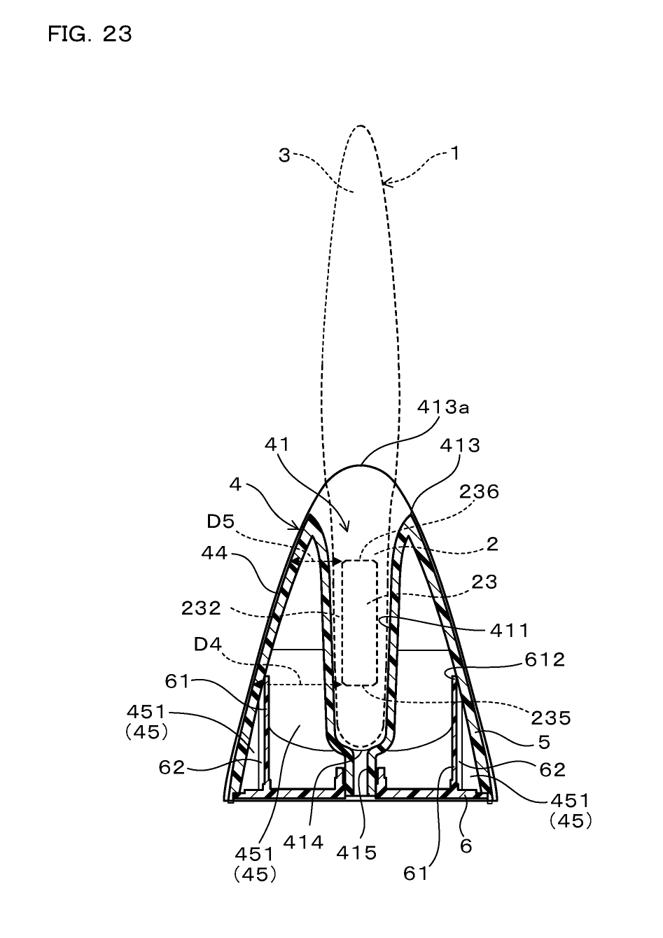

A distance D4 from the end faces 232 of the magnet 23 to the side surface 44 of the remover stand 4, as shown in FIG. 23, measured on the straight line extending from a lower end 235 of the magnet 23 in its thickness direction is 21.8 mm. A distance D5 from the end face 232 of the magnet 23 to the side surface 44 of the remover stand 4 measured on the straight line extending from an upper end 236 of the magnet 23 in its thickness direction is 12.6 mm.

When the remover 1 is held in the remover stand 4 of the present embodiment, the magnetic flux density generated by the magnet 23 is 80 mT or less at any position on the side surface 44 of the remover stand 4.

As shown FIGS. 22 and 23, the remover stand 4 is composed of an upper part 5 constituting the side surface 44, a top part 43 and the holding hollow part 41, and a lower part 6 constituting the bottom surface 42. The upper part 5 is open downward, and the lower part 6 is attached to the opening end of the upper part 5.

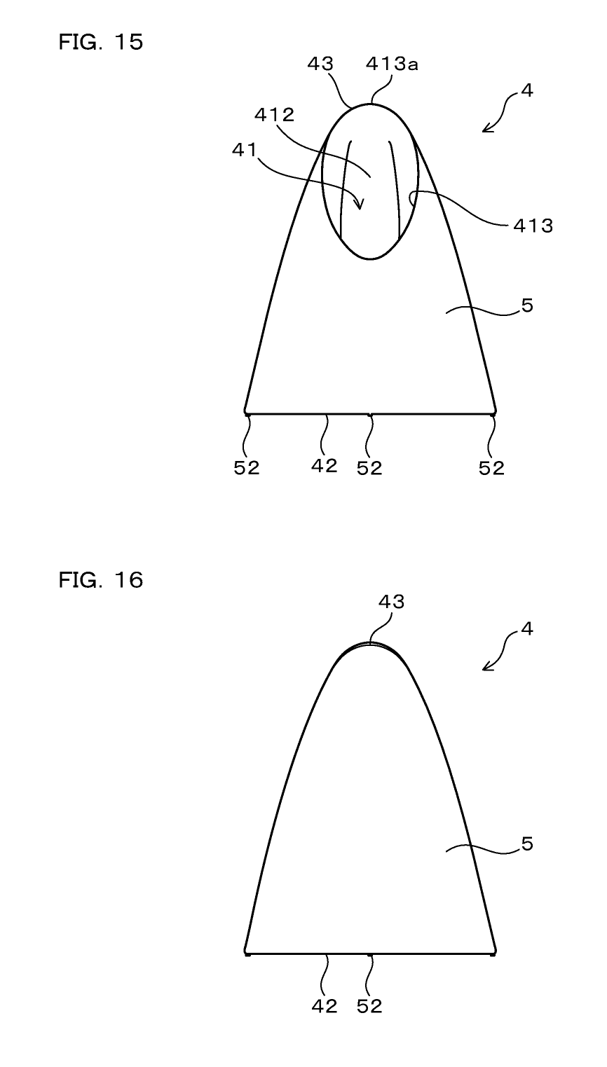

As shown in FIG. 21, the upper part 5 has six ribs 51 formed inside thereof, each radially extending from the holding hollow part 41. As shown in FIGS. 15, 16 and 20, the upper part 5 has projections 52 at three locations at the lower end edge thereof. When the remover stand 4 is placed on, for example, a desk, a table, or the like, the remover stand 4 is supported by these projections 52. In the state that the remover stand 4 is placed on the desk, the table, or the like, these projections 52 produce a space between the bottom surface 42 and the desk, the table or the like. A height h shown in FIG. 18, the height of the projection 52 of the remover stand 4 is 0.8 mm.

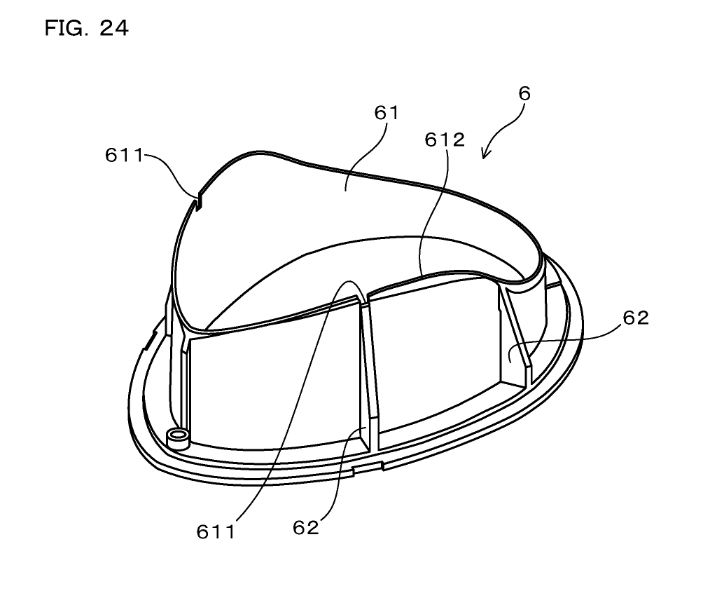

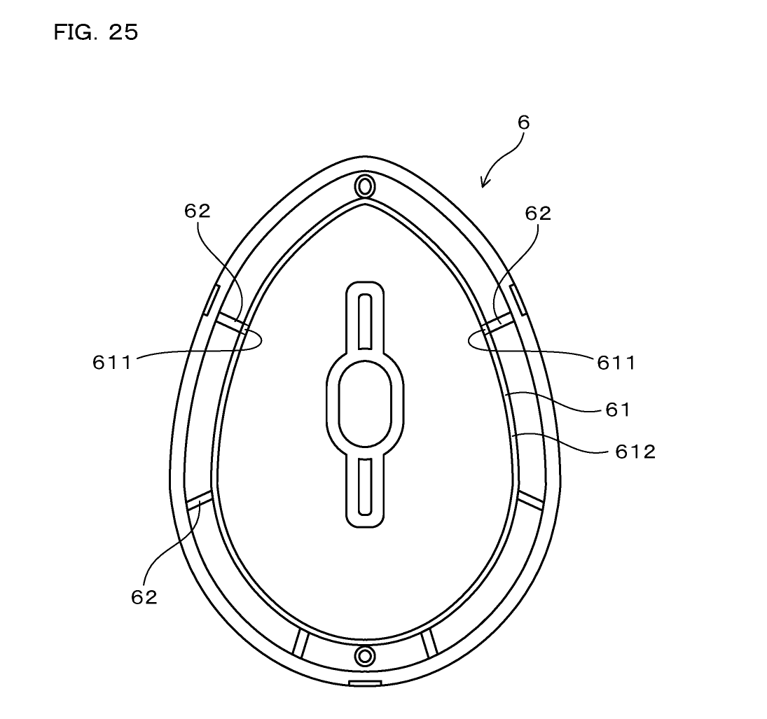

The lower part 6, as shown in FIG. 20, has an approximately flat elliptical shape that corresponds to the opening shape of the upper part 5. As shown in FIGS. 24 and 25, the lower part 6 has an annular rib 61 formed standing upward at an outer peripheral part thereof, and six radial ribs 62 formed radially extending from the annular rib 61. The annular rib 61 has notches 611 formed to avoid a collision with the rib of the upper part 5. As shown in FIG. 23, an upper end 612 of the annular rib 61 and the upper end of the radial rib 62 are extended to the vicinity of the inside surface of the upper part 5, and a slight clearance is produced therebetween.

Next, operational effects of the remover set 100 of the present embodiment will be described. The remover 1 in the remover set 100, as shown in FIG. 5, includes the recessed portion 311 recessed inward than the vicinity of the recessed portion 311 between the grip part 3 and the attraction head 2. The remover stand 4, as shown in FIG. 22, has the holding hollow part 41 configured to house and hold the attraction head 2 of the remover 1. The holding hollow part 41 includes the tip-holding portion 411 that has a concave shape corresponding to a shape including a tip of the attraction head 2 taking the holding posture, the opening end part 413 that is formed upward of the tip-holding portion 411, the guide part 412 that is arranged in a portion that is positioned between the tip-holding portion 411 and the opening end 413 and faces the recessed portion 311, and is inclined inward as going upward from the tip-holding portion 411.

In the remover set 100, the remover 1 can be placed in the remover stand 4, for instance, in the following way. Firstly, a user inserts the tip portion 13 of the remover 1 into the holding hollow part 41 from the opening end part 413, and then releases its hand(s) from the grip part 3 of the remover 1 to allow the remover 1 to drop down by its own weight. At that time, if the remover 1 takes the holding posture, the tip portion 13 of the remover 1 can reach the bottom of the tip-holding portion 411 as shown in FIG. 22.

As shown in FIG. 26, when the remover 1 takes a posture such that the grip part 3 is inclined to a recessed portion 311 side (i.e. the S-shaped curved portion 12 side), the tip portion 13 of the remover 1 is inserted into the holding hollow part 41 in the following way. Firstly, the lower end of the attraction head 2 in the holding posture (i.e. a part 22c constituting the tip portion 13 in the peripheral wall portion 22) is made abut on the part 411b of the tip-holding portion 411 corresponding to the mountain-shaped curved portion 11. Then, the remover 1 is stopped from going downward by friction resistance generated when the part 22c slides on the part 411b, the remover 1 is inclined largely to the S-shaped curved portion 12 side. As a result, a part 22a constituting the S-shaped curved portion 12 in the peripheral wall portion 22 is made abut on the guide part 412 as shown in FIG. 26.

At that time, because the guide part 412 bulges outward as going toward the lower end 412a from the upper end 412b, there is an allowance for the attraction head 2 to move downward. Consequently, the lower end part (i.e. the tip portion 13) of the remover 1 taking the holding posture can be moved along the part 411b that faces the mountain-shaped curved portion 11 in the tip-holding portion 411 (refer to an arrow 130). In this way, the tip portion 13 of the remover 1 can naturally reach the bottom of the tip-holding portion 411.

As shown in FIG. 27, when the remover 1 takes a posture such that the grip part 3 is inclined to the side opposite to the recessed portion 311 side (i.e. the mountain-shaped curved portion 11 side), the tip portion 13 of the remover 1 is inserted into the holding hollow part 41 in the following way. Firstly, the surface of the attraction head 2 on the opposite side to the recessed portion 311 (a part 22b constituting the mountain-shaped curved portion 11 in the peripheral wall portion 22) is made abut on the enlarged part 416 in the holding hollow part 41. Then, the mountain-shaped curved portion 11 slides on the enlarged part 416, so that the part 22a constituting the S-shaped curved portion 12 in the peripheral wall portion 22 is made abut on the guide part 412 as shown in FIG. 27.

At that time, because the guide part 412 bulges outward as going toward the lower end 412a from the upper end 412b, there is an allowance for the attraction head 2 to move downward. Consequently, the lower end part (i.e. the tip portion 13) of the remover 1 taking the holding posture can be made abut on the part 411b that faces the mountain-shaped curved portion 11 in the tip-holding portion 411, and can be slided therealong (refer to an arrow 131). In this way, the tip portion 13 of the remover 1 can naturally reach the bottom of the tip-holding portion 411.

As described above, in the remover set 100, the attraction head 2 of the remover 1 can easily reach the bottom of the holding hollow part 41 of the remover stand 4 even when the remover 1 takes the posture inclined from the holding posture to the S-shaped curved portion 12 side or the mountain-shaped curved portion 11 side. Consequently, in the remover set 100, it is possible to reduce work frequency in correcting the posture of the remover 1 by a user when inserting the remover 1 into the remover stand 4. As a result, the usability of the remover set 100 for the user can be improved.

As shown in FIG. 22, the tip-holding portion 411 of the holding hollow part 41 has the tip clearance part 414 dented downward than its vicinity at a position that corresponds to the tip portion 13 of the remover 1. Accordingly, it is possible to make the tip portion 13 of the remover 1, which is inserted into the holding hollow part 41, project downward from the tip clearance part 414. Thus, it is possible to position the attraction head 2 inside the holding hollow part 41, and to settle the posture of the remover 1 more stably.

The opening end part 413 has a sloping shape in which a height of the opening end part 413 on the side 413a corresponding to the S-shaped curved portion 12 is higher than that on the side 413b corresponding to the mountain-shaped curved portion 11. Accordingly, the guide part 412 can be formed over a larger area. It is possible to make the part 22a constituting the S-shaped curved portion 12 and the part 22c constituting the tip portion 13 in the peripheral wall surface 22 more easily abut on the guide part 412 after the attraction head 2 has been inserted into the holding hollow part 41. As a result, the tip portion 13 of the remover 1 can easily reach the bottom of the tip-holding portion 411.

The holding hollow part 41 has the enlarged part 416 between the part 411b of the tip-holding portion 411, which faces the mountain-shaped curved portion 11, and the opening end part 413. The enlarged part 416 has a shape of being enlarged as going upward. Accordingly, it is possible to make the mountain-shaped curved portion 11 and the enlarged part 416 abut on each other after inserting the attraction head 2 into the holding hollow part 41, and smoothly slide the mountain-shaped curved portion 11 along the enlarged part 416, for example, as shown in FIG. 27. In this way, it is possible to insert the tip portion 13 of the remover 1 so as to easily reach the bottom of the tip-holding portion 411.

The remover 1 includes the attraction head 2 including the pair of main wall portions 21, the peripheral wall portion 22 that connects peripheral edges of the pair of main wall portions 21 with each other, and the magnet 23 that is arranged between the pair of main wall portions 21. In addition, as shown in FIGS. 5 and 10, the attraction head 2 has the cosmetic agent wiping-off region 25 at the lower end part in the state that the remover 1 takes the posture, i.e. the posture such that the grip part 3 is directed upward and the attraction head 2. The cosmetic agent wiping-off region 25 generates a magnetic attraction force weaker than that generated in the vicinity of the cosmetic agent wiping-off region 25. Accordingly, as shown in FIG. 13, it is possible to collect the used cosmetic agent A adhered to the surface of the attraction head 2 together on the cosmetic agent wiping-off region 25 as shown in an arrow 250 and to easily wipe off it from the surface of the cosmetic agent wiping-off region 25. In this way, the remover 1 makes it possible to easily remove the cosmetic agent A from the surface of the attraction head 2.

In addition, the surface of the cosmetic agent wiping-off region 25 is provided at the specific position, that is, at the lower end part of the attraction head 2. Therefore, the user can collect the used cosmetic agent A together on the cosmetic agent wiping-off region 25 without regripping the grip part 3, and wipe off it from the attraction head 2. Thus, the remover 1 makes it possible to remove the used cosmetic agent A from the attraction head 2 more easily than conventional removers.

Further, the lower half part of the attraction head 2 of the present embodiment under taking the holding posture has a tapered shape the width of which is gradually tapered as going forward as shown in FIG. 10, and the cosmetic agent wiping-off region 25 is provided at the portion having the tapered shape. Therefore, it is possible to wipe off the cosmetic agent A collected together on the cosmetic agent wiping-off region 25 more easily.

The magnet 23 is placed in the attraction head 2 and arranged at the position apart from the inside surface 251 of the cosmetic agent wiping-off region 25 in the peripheral wall portion 22. Accordingly, the cosmetic agent wiping-off region 25 can be formed at the lower end part of the remover 1. Because there is no need for other components such as a soft-magnetic material, or the like to be attached in forming the cosmetic agent wiping-off region 25, it is possible to avoid increase in number of parts and simplify assembly work for the remover 1.

As shown in FIGS. 5 and 9, under the remover 1 taking the holding posture, the lower half part of the remover 1 includes the mountain-shaped curved portion 11 having a convexly curved contour on the left side in a plan view on either one of the pair of main wall portions 21. In addition, on the right side in the plan view, the lower half part of the remover 1 includes the S-shaped curved portion 12 having an S-shaped contour that is recessed inward, expands outward, and is recessed inward again as going downward from upward. Further, the lower half part of the remover 1 includes the tip portion 13 that is provided between the mountain-shaped curved portion 11 and the S-shaped curved portion 12 and has a convexly curved contour with a curvature radius smaller than that of any portion of the mountain-shaped curved portion 11 and the S-shaped curved portion 12.

According to such a configuration, the grip part 3 has a shape to be easily fitted to user's hand, so that the posture of the remover 1 can be easily stabilized in the removal work of the used cosmetic agent A from the human skin. In addition, as shown in FIG. 12, if the attraction head 2 is brought to the human skin in such a manner to allow the S-shaped curved portion 12 side to be closer to the cosmetic agent A, it is possible to make the grip part 3 hardly collide with a human body. As a result, it is made easy for the user to attract the used cosmetic agent A and remove it from all over the human skin. Thus, the usability of the remover 1 in the removal work of the used cosmetic agent A from the human skin can be improved.

The remover stand 4 of the present embodiment has the projections 52 at the lower end edge of the upper part 5 to support the remover stand 4. Such a configuration makes it possible to prevent the remover stand 4 from sticking to the desk or the like due to the surface tension of any liquid such as water entering into the space between the bottom surface 42 of the remover stand 4 and the desk or the like.

On the bottom surface 42 of the remover stand 4, the cylindrical part 415 is opened. When any liquid such as water enters into the space between the bottom surface 42 of the remover stand 4 and the desk or the like, such a configuration makes it possible to attract the liquid into the cylindrical part 415 by the surface tension of the liquid. As a result, it is possible to prevent the remover stand 4 from sticking to the desk or the like due to the surface tension.

As shown in FIGS. 24 and 25, the lower part 6 of the remover stand 4 includes the annular rib 61 and the radial ribs 62 each formed standing upward. These ribs enhance the rigidity of the remover stand 4, so that the vibration generated when the remover 1 abuts on the holding hollow part 41 can be damped. As a result, sounds generated when the remover 1 abuts on the holding hollow part 41 can be reduced.

As shown in FIG. 23, an internal space 45 in the remover stand 4, i.e. the space formed between the upper part 5 and the lower part 6 is divided into a plurality of small spaces 451 by the annular rib 61 and the radial ribs 62. Such a configuration make it possible to reduce echoes of the sounds generated when the remover 1 abuts on the holding hollow part 41 and reduce the sounds generated when the remover 1 abuts on the holding hollow part 41. In addition, it is possible to control the frequency of the sounds generated when the remover 1 abuts on the holding hollow part 41 and prevent generation of sounds uncomfortable for the user.

Aspects of the remover 1 and the remover set 100 according to the present invention are not limited to the aforementioned embodiment, and various modification are possible within a range that does not deviate from the gist of the present invention. For example, the arrangement of the magnet 23 in the remover 1 may be modified as appropriate in accordance with the shape and/or magnitude of magnetic flux density of the magnet 23. The distance from the side peripheral surface 233 of the magnet 23 to the cosmetic agent wiping-off region 25 may be longer or shorter than the distances L1 in the embodiment as long as the magnetic flux density in the cosmetic agent wiping-off region 25 can be sufficiently reduced, or can be reduced to zero.

In the remover stand 4, the distance D1 from the magnet 23 to the bottom surface 42, the distances D1 to D5 from the magnet 23 to the side surface 44 may be longer or shorter than the distances in the embodiment as long as the magnetic flux density in the bottom surface 42 and the side surface 44 in the remover stand 4 can be sufficiently reduced. The projections 52 may be formed on the upper part 5 or on the lower part 6. In order to stabilize the posture of the remover stand 4, the projection 52 has only to be formed at least at three locations.

The embodiment shows an example of the remover 1 having the cosmetic agent wiping-off region 25 that generates a magnetic attraction force weaker than that generated in the vicinity thereof. However, the cosmetic agent wiping-off region 25 may generate no magnetic attraction force.

1. A remover set comprising:
a remover that comprises:
an attraction head that has a magnet built therein and is configured to attract a cosmetic agent that has been applied on a human skin by a magnetic attraction force, thereby removing the cosmetic agent from the human skin;
a root part that connects to the attraction head; and
a grip part that extends from the root part; and
a remover stand configured to keep the remover taking a holding posture such that the grip part is directed upward and the attraction head is directed downward; wherein
the root part of the remover has a recessed portion recessed inward than a vicinity of the recessed portion,
the remover stand has a holding hollow part configured to house and hold the attraction head of the remover,
the holding hollow part comprises:
a tip-holding portion that is formed inside of the remover stand and has a concave shape corresponding to a shape including a tip of the attraction head of the remover taking the holding posture;
an opening end part that is formed upward of the tip-holding portion, from which the attraction head is inserted; and
a guide part that is arranged in a portion that is positioned between the tip-holding portion and the opening end part and faces the recessed portion, and is inclined inward as going upward from the tip-holding portion.

2. The remover set according to claim 1, wherein the tip-holding portion of the remover stand has a tip clearance part dented downward than a vicinity thereof at a position that corresponds to the tip of the attraction head of the remover taking the holding posture.

3. The remover set according to claim 1, wherein the attraction head of the remover has a cosmetic agent wiping-off region at a lower end part thereof under the remover taking the holding posture, the cosmetic agent wiping-off region generating a magnetic attraction force weaker than that generated in a vicinity of the cosmetic agent wiping-off region or generating no magnetic attraction force.

4. The remover set according to claim 2, wherein the attraction head of the remover has a cosmetic agent wiping-off region at a lower end part thereof under the remover taking the holding posture, the cosmetic agent wiping-off region generating a magnetic attraction force weaker than that generated in a vicinity of the cosmetic agent wiping-off region or generating no magnetic attraction force.

5. The remover set according to claim 1, wherein
the attraction head of the remover comprises:
a pair of main wall portions facing each other;
a peripheral wall portion that connects peripheral edges of the pair of main wall portions with each other; and
the magnet that is arranged between the pair of main wall portions such that an inside surface of each main wall portion faces a magnet pole,
a lower half part of the remover under the remover taking the holding posture comprises:
a mountain-shaped curved portion having a convexly curved contour on a left side in a plan view on one of the pair of main wall portions;
an S-shaped curved portion having an S-shaped contour that is recessed inward, expands outward, and is recessed inward again as going downward from upward on a right side in the plan view; and
a tip portion that is provided between the mountain-shaped curved portion and the S-shaped curved portion and has a convexly curved contour with a curvature radius smaller than that of any portion of the mountain-shaped curved portion and the S-shaped curved portion,
the tip-holding portion of the holding hollow part of the remover stand has a hollowed shape corresponding to a shape including a lower half part of the mountain-shaped curved portion, a lower half part of the S-shaped curved portion, and the tip portion as a lower end of the remover taking the holding posture, and
the guide part is arranged between a part of the tip-holding portion, which faces the S-shaped curved portion, and the opening end part.

6. The remover set according to claim 2, wherein
the attraction head of the remover comprises:
a pair of main wall portions facing each other;
a peripheral wall portion that connects peripheral edges of the pair of main wall portions with each other; and
the magnet that is arranged between the pair of main wall portions such that an inside surface of each main wall portion faces a magnet pole,
a lower half part of the remover under the remover taking the holding posture comprises:
a mountain-shaped curved portion having a convexly curved contour on a left side in a plan view on one of the pair of main wall portions;
an S-shaped curved portion having an S-shaped contour that is recessed inward, expands outward, and is recessed inward again as going downward from upward on a right side in the plan view; and
a tip portion that is provided between the mountain-shaped curved portion and the S-shaped curved portion and has a convexly curved contour with a curvature radius smaller than that of any portion of the mountain-shaped curved portion and the S-shaped curved portion,
the tip-holding portion of the holding hollow part of the remover stand has a hollowed shape corresponding to a shape including a lower half part of the mountain-shaped curved portion, a lower half part of the S-shaped curved portion, and the tip portion as a lower end of the remover taking the holding posture, and
the guide part is arranged between a part of the tip-holding portion, which faces the S-shaped curved portion, and the opening end part.

7. The remover set according to claim 3, wherein
the attraction head of the remover comprises:
a pair of main wall portions facing each other;
a peripheral wall portion that connects peripheral edges of the pair of main wall portions with each other; and
the magnet that is arranged between the pair of main wall portions such that an inside surface of each main wall portion faces a magnet pole,
a lower half part of the remover under the remover taking the holding posture comprises:
a mountain-shaped curved portion having a convexly curved contour on a left side in a plan view on one of the pair of main wall portions;
an S-shaped curved portion having an S-shaped contour that is recessed inward, expands outward, and is recessed inward again as going downward from upward on a right side in the plan view; and
a tip portion that is provided between the mountain-shaped curved portion and the S-shaped curved portion and has a convexly curved contour with a curvature radius smaller than that of any portion of the mountain-shaped curved portion and the S-shaped curved portion,
the tip-holding portion of the holding hollow part of the remover stand has a hollowed shape corresponding to a shape including a lower half part of the mountain-shaped curved portion, a lower half part of the S-shaped curved portion, and the tip portion as a lower end of the remover taking the holding posture, and
the guide part is arranged between a part of the tip-holding portion, which faces the S-shaped curved portion, and the opening end part.

8. The remover set according to claim 4, wherein
the attraction head of the remover comprises:
a pair of main wall portions facing each other;
a peripheral wall portion that connects peripheral edges of the pair of main wall portions with each other; and
the magnet that is arranged between the pair of main wall portions such that an inside surface of each main wall portion faces a magnet pole,
a lower half part of the remover under the remover taking the holding posture comprises:
a mountain-shaped curved portion having a convexly curved contour on a left side in a plan view on one of the pair of main wall portions;
an S-shaped curved portion having an S-shaped contour that is recessed inward, expands outward, and is recessed inward again as going downward from upward on a right side in the plan view; and
a tip portion that is provided between the mountain-shaped curved portion and the S-shaped curved portion and has a convexly curved contour with a curvature radius smaller than that of any portion of the mountain-shaped curved portion and the S-shaped curved portion,
the tip-holding portion of the holding hollow part of the remover stand has a hollowed shape corresponding to a shape including a lower half part of the mountain-shaped curved portion, a lower half part of the S-shaped curved portion, and the tip portion as a lower end of the remover taking the holding posture, and
the guide part is arranged between a part of the tip-holding portion, which faces the S-shaped curved portion, and the opening end part.

9. The remover set according to claim 5, wherein the holding hollow part of the remover stand has an enlarged part between a part of the tip-holding portion, which faces the mountain-shaped curved portion, and the opening end part, the enlarged part having a shape of being enlarged as going upward.

10. The remover set according to claim 6, wherein the holding hollow part of the remover stand has an enlarged part between a part of the tip-holding portion, which faces the mountain-shaped curved portion, and the opening end part, the enlarged part having a shape of being enlarged as going upward.

11. The remover set according to claim 7, wherein the holding hollow part of the remover stand has an enlarged part between a part of the tip-holding portion, which faces the mountain-shaped curved portion, and the opening end part, the enlarged part having a shape of being enlarged as going upward.

12. The remover set according to claim 8, wherein the holding hollow part of the remover stand has an enlarged part between a part of the tip-holding portion, which faces the mountain-shaped curved portion, and the opening end part, the enlarged part having a shape of being enlarged as going upward.

13. The remover set according to claim 5, wherein the opening end part in the remover stand has a sloping shape in which a height of the opening end part on a side corresponding to the S-shaped curved portion is higher than that on a side corresponding to the mountain-shaped curved portion.

14. The remover set according to claim 6, wherein the opening end part in the remover stand has a sloping shape in which a height of the opening end part on a side corresponding to the S-shaped curved portion is higher than that on a side corresponding to the mountain-shaped curved portion.

15. The remover set according to claim 7, wherein the opening end part in the remover stand has a sloping shape in which a height of the opening end part on a side corresponding to the S-shaped curved portion is higher than that on a side corresponding to the mountain-shaped curved portion.

16. The remover set according to claim 8, wherein the opening end part in the remover stand has a sloping shape in which a height of the opening end part on a side corresponding to the S-shaped curved portion is higher than that on a side corresponding to the mountain-shaped curved portion.

17. The remover set according to claim 9, wherein the opening end part in the remover stand has a sloping shape in which a height of the opening end part on a side corresponding to the S-shaped curved portion is higher than that on a side corresponding to the mountain-shaped curved portion.

18. The remover set according to claim 10, wherein the opening end part in the remover stand has a sloping shape in which a height of the opening end part on a side corresponding to the S-shaped curved portion is higher than that on a side corresponding to the mountain-shaped curved portion.

19. The remover set according to claim 11, wherein the opening end part in the remover stand has a sloping shape in which a height of the opening end part on a side corresponding to the S-shaped curved portion is higher than that on a side corresponding to the mountain-shaped curved portion.

20. The remover set according to claim 12, wherein the opening end part in the remover stand has a sloping shape in which a height of the opening end part on a side corresponding to the S-shaped curved portion is higher than that on a side corresponding to the mountain-shaped curved portion.
